(19) United States
(12) Patent Application Publication
(10) Pub. No.: US 2004/0022833 A1
(43) Pub. Date: Feb. 5, 2004

(54) Child's wiping system

(76) Inventors: Jill Hartwig, Glendale, AZ (US); Kalen Brown, Phoenix, AZ (US)

Correspondence Address: Jill Hartwig, 6030 W. Kerry Lane, Glendale, AZ, 85308, US

(21) Appl. No.: 10/628,845

(22) Filed: Jul. 26, 2003

Related U.S. Application Data
(60) Provisional application No. 60/400,633, filed on Aug. 2, 2002.

(51) Int. Cl. A61K 9/70

(52) U.S. Cl. 424/443

(57) ABSTRACT

A system for aiding children in wiping and self-cleaning after toilet use. More specifically, a disposable “glove”-like sheath into which the child's hand is inserted and is then used to remove urine and fecal waste. After use, the glove is preferably deposited in the toilet. The sheath consists of a folded and seamed pre-moistened, non-woven material that can include a water dispersible binder. A dispenser system for the child's hand-wearable wiping system is also disclosed.

This application claims the benefit under 35USC119 (E) of U.S. Provisional Application No. 60/400,633 filed Aug. 2^{nd}, 2002.

### BACKGROUND

This invention relates to providing an improved system for aiding children in wiping and self-cleaning after toilet use. More specifically, this invention relates to a disposable “glove”-like sheath into which the child's hand is inserted, which sheath is used for removal and cleaning of urine and fecal waste. After use, the sheath is preferably deposited in the toilet.

Typically, young children find difficulty in successfully wiping away urine and fecal material using dry toilet tissue. Many young children discover that dispensing and holding the tissue is a difficult skill that often results in messy hands, clothes and surrounding surfaces. The child's attempt to hold the tissue is a proper orientation for wiping is often haphazard at best. Young children characteristically grip the tissue in a tightly balled fist, leaving as much of the wiping hand exposed as cleaning tissue. Dry tissue, although used extensively in the United States and internationally, is not particularly efficient at removing bodily wastes, especially fecal material. Unsuccessful attempts at hygienic post cleaning can cause embarrassment and stress for both the young and supervising adult. Additionally, poor hygienic cleaning can lead to the spread of disease when soiled hands and clothing transfer bodily waste to common areas.

A need exists for a convenient wiping accessory that is simple to use by a child and provides increased ease of cleaning and improved protection for the child's hand.

### OBJECTS OF THE INVENTION

A primary object and feature of the present invention is to provide a system for aiding children in wiping and self-cleaning to remove urine and fecal waste. It is a further object and feature of the present invention to provide a system that is configured and sized for use by a young child. It is another object and feature of the present invention to provide such a system that protectively covers the child's hand. It is an additional object and feature of the present invention to provide a system that utilizes disposable and flushable materials. It is another object and feature of the present invention to provide such a system that is pre-moistened to aide in hygienic cleaning. It is yet another object and feature of the present invention to provide such a system that comprises a plurality of wipes joined to form a dispensable grouping. It is a further object and feature of the present invention to provide a range of refillable dispensers having playful indicia. Another primary object and feature of the present invention is to provide such a system that is efficient, inexpensive, and handy. Other objects and features of this invention will become apparent with reference to the following descriptions.

### SUMMARY OF THE INVENTION

In accordance with a preferred embodiment hereof, this invention provides a child's disposable wiping system to remove urine and feces after the child's elimination of bodily waste, hand-wearable by the child, comprising in combination: at least one hand-wearable cover structured and arranged to substantially cover a child's hand; at least one pre-moisturizer structured and arranged to pre-moisturize such at least one hand-wearable cover; wherein such at least one hand-wearable cover is fluid permeable; wherein such at least one pre-moisturizer substantially impregnates such at least one hand-wearable cover. Moreover, it provides such a system wherein such at least one hand-wearable cover comprises at least one surface structured and arranged to retain the bodily waste during wiping. Additionally, it provides such a system wherein such hand-wearable cover comprises a single layer fabric. Also, it provides such a system wherein such single layer fabric comprises at least one non-woven material. In addition, it provides such a system wherein such single layer fabric comprises at least one water dispersible binder. And, it provides such a system wherein such at least one hand-wearable cover comprises: a first side portion; a second side portion; at least one opening wherein such first side portion and such second side portion are joined to define at least one interior portion; wherein such at least one opening is structured and arranged to permit the child's hand to be removably inserted into such at least one interior portion. Further, it provides such a system wherein such at least one hand-wearable cover further comprises: at least one grip assister structured and arranged to assist in gripping at least one peripheral portion of such at least one opening; whereby such grip assister facilitates separation of such first side portion from such second side portion to assist insertion of the child's hand through such opening of such hand-wearable cover. Even further, it provides such a system wherein such at least one grip assister comprises at least one scalloped-shaped edge along such at least one peripheral portion of such at least one opening. Moreover, it provides such a system wherein such at least one hand-wearable cover comprises at least one finger receiving portion structured and arranged to receive at least one finger of the child's hand. Even further, it provides such a system wherein such at least one hand cover comprises a thumb receiving portion structured and arranged to receive a thumb of the child's hand. Further, it provides such a system wherein: a plurality of such hand-wearable covers are detachably joined to form at least one continuous band; wherein such at least one continuous band is in a rolled configuration. Even further, it provides such a system wherein such at least one continuous band comprises at least one perforation structured and arranged to assist the user in detaching at least one hand-wearable cover from such at least one band. Additionally, it provides such a system wherein a plurality of such at least one hand-wearable covers are interfolded to form a dispensable stack.

In accordance with another preferred embodiment hereof, this invention provides a method of making at least one child's hand-wearable wipe comprising the steps of: receiving a quantity of desired fabric material; reducing such fabric material to a desired working width; cutting such fabric to produce at least one continuous sheet of a desired width; folding such at least one continuous sheet to form at least one continuous, essentially “U”-shaped band; seaming such at least one continuous, essentially “U”-shaped band to form at least one child's hand-wearable wipe. The method of making at least one child's hand-wearable wipe further comprising the step of applying to such at least one continuous, essentially “U”-shaped band, at least one line of perforations located essentially perpendicular to the longitudinal axis of such at least one continuous, essentially “U”-shaped band. In addition, it provides such a method of making at least one child's hand-wearable wipe further comprising the step of cutting such at least one continuous, essentially “U”-shaped band to produce at least one child's hand-wearable wipe. Even further, it provides such a method of making at least one child's hand-wearable wipe further comprising the step of folding such at least one child's hand-wearable wipe to form a dispensable stack. Further, it provides such a method of making at least one child's hand-wearable wipe, further comprising the step of impregnating such at least one child's hand-wearable wipe with at least one moisturizing fluid.

In accordance with another preferred embodiment hereof, this invention provides a child's hand-wearable wipe dispenser system adapted to dispense at least one child's hand-wearable wipe comprising: at least one holder structured and arranged to hold at least one child's hand-wearable wipe; wherein such at least one holder comprises: at least one housing having at least one interior portion structured and arranged to house the at least one child's hand-wearable wipe; at least one dispenser structured and arranged to dispense the at least one child's hand-wearable wipe exterior of such housing; at least one portal structured and arranged to allow access to such at least one interior portion; at least one moisture retainer structured and arranged to retain moisture within such at least one interior portion; wherein the size and configuration of such at least one holder closely conforms to the dispensable format of the at least one child's hand-wearable wipe; wherein such at least one holder comprises indicia. Further, it provides such a system wherein such at least one holder is supported by a dry rolled tissue dispenser. Moreover, it provides each and every novel feature, element, combination, step and/or method disclosed or suggested by this provisional patent application.

### BRIEF DESCRIPTION OF THE DRAWINGS

FIG. 1 is a perspective view of a child's wiping system according to a preferred embodiment of the present invention.

FIG. 2 is a perspective view of the child's wiping system engaging a child's hand according to the embodiment of FIG. 1.

FIG. 3 is a front view of the child's wiping system according to the embodiment of FIG. 1.

FIG. 4 is the sectional view 4-4 of FIG. 3 through the child's wiping system.

FIG. 5 is the sectional view 5-5 of FIG. 3 through the child's wiping system.

FIG. 6 is a front view of a multiple series of child's wipes according to the embodiment of FIG. 1.

FIG. 7 is a perspective view of the child's wiping system in a rolled configuration according to the embodiment of FIG. 6.

FIG. 8 is a perspective view of the child's wiping system in a folded configuration according to the embodiment of FIG. 1.

FIG. 9 is a perspective view of a folded product dispenser for the child's wiping system according to another preferred embodiment of the present invention.

FIG. 10 is a perspective view of an alternate folded product dispenser for the child's wiping system according to the embodiment of FIG. 9.

FIG. 11 is a perspective view of a tubular dispenser for the child's wiping system according to another preferred embodiment of the present invention.

FIG. 12 is a perspective view of a hanging dispenser for the rolled child's wiping system, adapted to hang from a standard dry tissue dispenser according to another preferred embodiment of the present invention.

FIG. 13 is a perspective view of the hanging dispenser for the child's wiping system in an open position to receive a wiping sheath roll according to the embodiment of FIG. 12.

FIG. 14 is a perspective view of a hanging dispenser holding a stack of interfolded wiping sheaths according to another preferred embodiment of the present invention.

FIG. 15 is a perspective view of the hanging dispenser in an open position according to the embodiment of FIG. 14.

FIG. 16 is a perspective view of a preferred step in the manufacture of a child's wipe according to the embodiment of FIG. 1.

FIG. 17 is a perspective view of another preferred step in the manufacture of a child's wipe according to the embodiment of FIG. 1.

FIG. 18 is a perspective view of another preferred step in the manufacture of a child's wipe according to the embodiment of FIG. 1.

FIG. 19 is a front view of an alternate arrangement of multiple child wipes according to the embodiment of FIG. 1.

FIG. 20 is a front view of an alternate preferred embodiment of the child's wipe.

### DETAILED DESCRIPTION OF A PREFERRED EMBODIMENT OF THE INVENTION

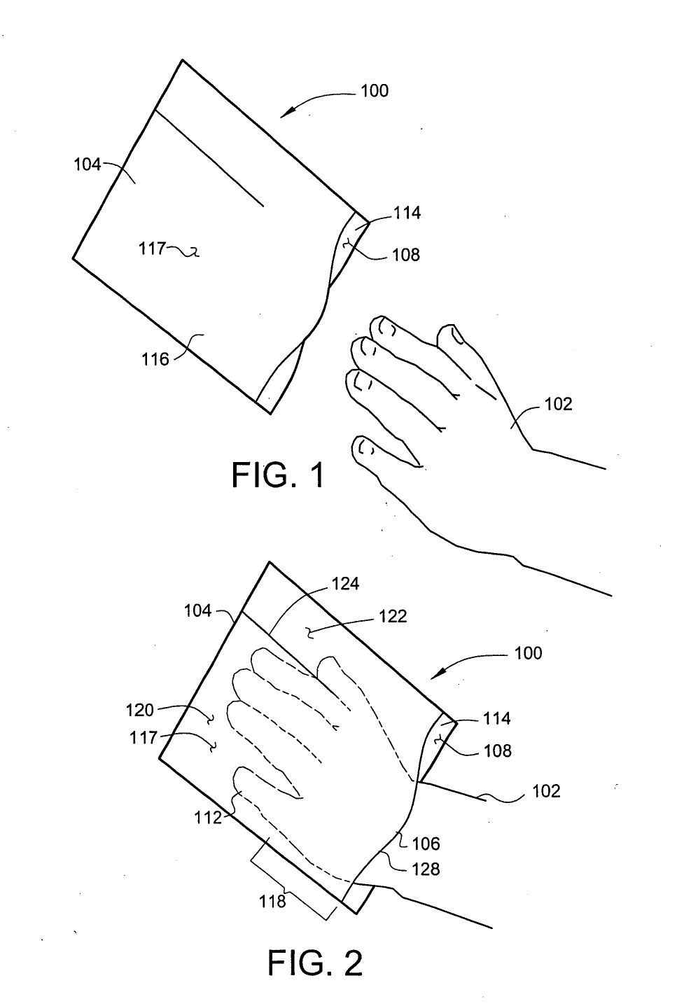

FIG. 1 is a perspective view of child's wiping system 100 according to a preferred embodiment of the present invention. Preferably, child's wiping system 100 consists of an essentially rectangular, pre-moistened, glove-like wiping sheath 104 into which child's hand 102 may be inserted (herein embodying at least one hand-wearable cover structured and arranged to substantially cover a child's hand). Wiping sheath 104 preferably consists of front panel 114, back panel 116, and opening 108, as shown (herein embodying wherein such at least one hand-wearable cover comprises: a first side portion; a second side portion; and at least one opening). Preferably, front panel 114 and back panel 116 are peripherally joined to form internal hand-receiving portion 117, as shown.

FIG. 2 is a perspective view of the child's wiping system 100 engaging child's hand 102 according to the preferred embodiment of FIG. 1. Grip-assisting scallop(s) 106 are preferably located at opening 108 to aid in separating the front face 114 from the back face 116, thereby allowing easy passage of child's hand 102 through opening 108 into hand-receiving portion 117 of child's wiping system 100 (herein embodying wherein such at least one opening is structured and arranged to permit the child's hand to be removably inserted into such at least one interior portion and further embodying wherein such at least one hand-wearable cover further comprises: at least one grip assister structured and arranged to assist in gripping at least one peripheral portion of such at least one opening; whereby such grip assister facilitates separation of such first side portion from such second side portion to assist insertion of the child's hand through such opening of such hand-wearable cover; wherein such at least one grip assister comprises at least one scalloped-shaped edge along such at least one peripheral portion of such at least one opening), as shown. Preferably, internal hand-receiving portion 117 of wiping sheath 104 is subdivided into finger receiving portion 120 and thumb receiving portion 122 by divider seam 124, as shown (herein embodying wherein such at least one hand-wearable cover comprises at least one finger receiving portion structured and arranged to receive at least one finger of the child's hand and further embodying wherein such at least one hand cover comprises a thumb receiving portion structured and arranged to receive a thumb of the child's hand). Although less preferred, under appropriate circumstances, internal hand-receiving portion 117 may consist of a single undivided interior compartment (e.g., to produce a less costly version of the system).

It is known that the hand of a small child varies from that of an adult, not only in size, but also in relative proportion. On average, the fingers 112 of a child's hand are shorter in length relative to the child's palm portion 118 when proportionally compared to the hand of an adult. The proportions of wiping sheath 104 are preferably configured to accommodate the unique physical proportions of a small child's hand 102, as shown.

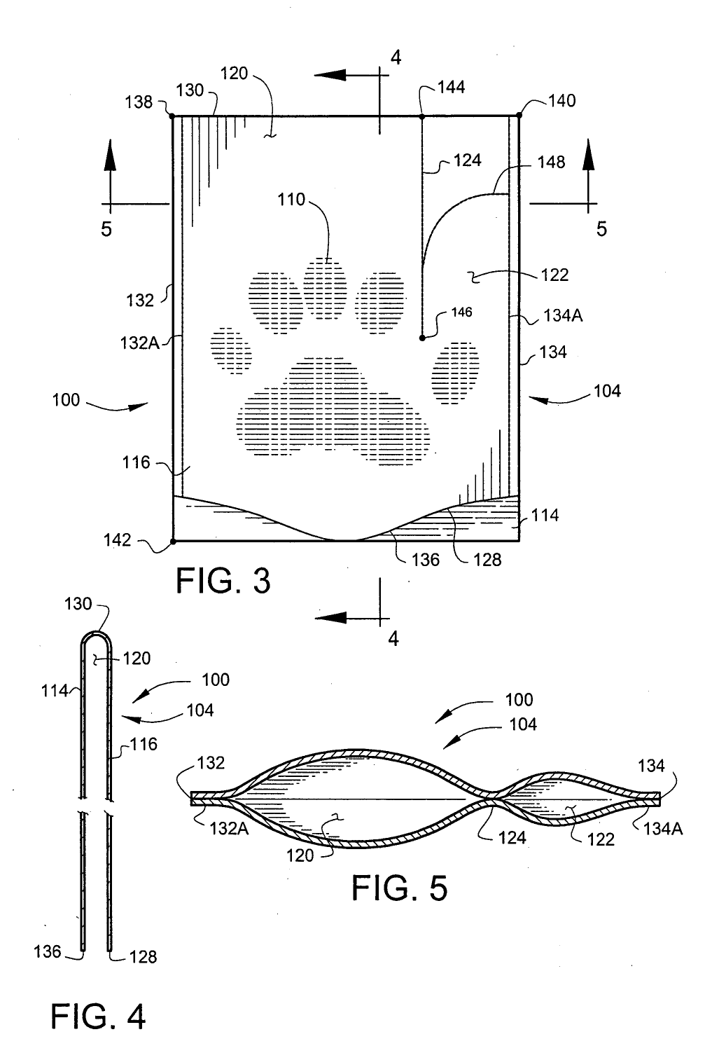

FIG. 3 is a front view of child's wiping system 100 according to the preferred embodiment of FIG. 1. As shown, the wiping sheath 104 preferably includes top edge 130, first side edge 132, second side edge 134, first bottom edge 128, and second bottom edge 136, as shown. Wiping sheath 104 is preferably made from a single sheet of fabric folded along top edge 130 to form front panel 114 and back panel 116.

Preferably, the material of wiping sheath 104 is a single ply, non-woven fabric (herein embodying wherein such hand-wearable cover comprises a single layer fabric). As used herein, the term “non-woven fabric” refers to a fabric composed of individual fibers randomly arranged in a mat-like structure bound by one or more physical or chemical binders. To provide a preferred combination of economy, absorbency and texture, the material of wiping sheath 104 is preferably a non-woven fabric produced by an air-laid process, primarily composed of cellulose fibers, having a basis weight ranging from about 30 to about 60 grams per square meter (herein embodying wherein such at least one hand-wearable cover comprises at least one surface structured and arranged to retain the bodily waste during wiping). Under appropriate circumstances, other commercially available non-woven materials not limited to polyolefin, polyester, and rayon fibers, wet-laid fabrics, hydro-entangled fabrics, staple-fiber carded and solution-spun fabrics may be used to construct child's wiping system 100. Preferably, the material of wiping sheath 104 is fluid-permeable (embodying herein wherein such at least one hand-wearable cover is fluid-permeable) and under appropriate circumstances may be multi-plied or quilted. It is highly preferred that the materials of wiping sheath 104 be flushable such that child's wiping system 100 may be placed in the toilet for disposal without risk of clogging sewer or septic systems. It is preferred that the binder of the non-woven material of wiping sheath 104 be dispersible in neutral or alkaline conditions, with limited dispersibility in ionic solutions, such as body fluids. The preferred characteristic of ion-sensitive dispersibility allows child's wiping system 100 to be effective in removing bodily waste while remaining safely flushable for convenience of use (herein embodying wherein such single layer fabric comprises at least one water dispersible binder). Commercially available products, such as water dispersible, non-woven fabrics produced by Kimberly-Clark Corporation of North America are suitable for the construction of wiping sheath 104.

Preferably, front panel 114 and back panel 116 are continuously joined along first side edge 132 and second side edge 134 to form internal hand-receiving portion 117 (herein embodying wherein such first side portion and such second side portion are joined to define at least one interior portion), as shown. Preferably, the side edges of front panel 114 and back panel 116 are positioned in a face-to-face relationship to-allow continuous edge seams, seam 132A and seam 134A to be formed, as shown. Similarly, divider seam 124 is preferably applied to join front panel 114 and back panel 116 to create finger receiving portion 120 and thumb receiving portion 122, as shown. Seaming of wiping sheath 104 is accomplished through gluing, sewing, heat sealing, pressure bonding, ultrasonic sealing, or combinations of the above systems. Under appropriate circumstances, other seaming methods compatible with non-woven fabrics may be used.

Preferably, bottom edge 128 is cut in a scalloped manner to facilitate gripping by the fingers of the user. Preferably, scallop 136 is cut to expose at least one portion of back panel 116. It should be noted that although FIG. 3 depicts a single scallop 136, under appropriate circumstances, a plurality of smaller conjoined scallops may suffice.

As previously noted in the description of FIG. 2, the arrangements of wiping sheath 104 are preferably configured to accommodate the unique physical proportions of the hand of a small child. Preferably, the width of wiping sheath 104 along top edge 130, from upper corner 138 to upper corner 140 is 5-½″. The preferred length of wiping sheath 104 along first side edge (and second side edge 134) from upper corner 138 to lower corner 142 is 6-¼″. The preferred width of thumb receiving portion 122 from upper corner 140 to upper endpoint 144 of divider seam 124 is 1-½″. Preferably, the length of divider seam 124 from upper endpoint 144 to lower endpoint 146 is about 3-½″. Under appropriate circumstances, second divider seam 148 may be applied to thumb receiving portion 122 to add additional strength along divider seam 124, to help guide the child's thumb into a proper position and aid the child in visually identifying the correct orientation of wiping sheath 104 prior to hand insertion. It should be noted that, under appropriate circumstances, the size of child's wiping system 100 may be larger or smaller to accommodate young children of various ages.

Preferably, wiping sheath 104 is printed with playful and/or instructive indicia 110 and may be color dyed. In the example of FIG. 3, a playfully rendered animal paw is imprinted on the exterior surface of wiping sheath 104. Because wiping sheath 104 is reversible for right and left hand use, indicia are preferably printed on both the front and rear of wiping sheath 104. Preferably, all materials used in child's wiping system 100, including inks and dyes, are non-toxic. Non-toxic, colorfast inks and dyes compatible with non-woven materials are commercially available, and as those skilled in the art will now appreciate, are suitable for application on child's wiping system 100. The most commonly employed methods of printing to non-woven fabrics are screen and rotary screen-printing, both of which are suitable for use with the present invention.

Wiping sheath 104 of child's wiping system 100 is preferably pre-moistened with at least one moisture-impregnating fluid consisting primarily of water (herein embodying at least one pre-moisturizer structured and arranged to pre-moisturize such at least one hand-wearable cover). Under appropriate circumstances, the water-based fluid is preferably modified with a number of performance improving and cosmetic admixtures, including humectants, preservatives and fragrances. A preferred group of the foregoing admixtures may, under appropriate circumstances, include humectants, such as propylene glycol, emulsifiers, such as disodium cocoamphodiacetate, and preservatives, such as DMDM Hydantoin and/or polysorbate 20. It is highly preferred that the water-based moisturizer completely impregnates the non-woven fabric of wiping sheath 104 to allow for sufficient moisturizer release during use (herein embodying wherein such at least one pre-moisturizer substantially impregnates such at least one hand-wearable cover).

FIG. 4 is the sectional view 4-4 of FIG. 3 through child's wiping system 100. As shown, wiping sheath 104 is preferably composed of a single sheet of fabric folded essentially in half along top edge 130 to form front panel 114, back panel 116, and finger receiving portion 120, as shown.

FIG. 5 is the sectional view 5-5 of FIG. 3 through child's wiping system 100. The preferred interior divisions of wiping sheath 104, consisting of finger receiving portion 120 and thumb receiving portion 122, are clearly seen in the section of FIG. 5. Contact areas between front panel 114 and back panel 116 at seam 132A, divider seam 124, and seam 134A are also shown in FIG. 5.

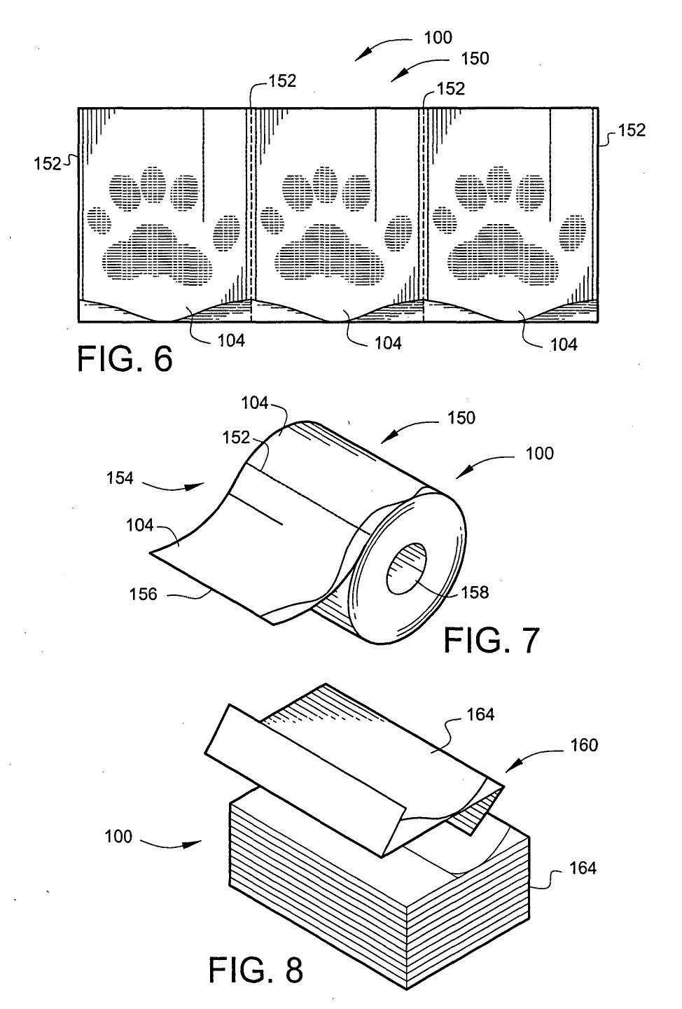

FIG. 6 is a front view of a multiple series of wiping sheath(s) 104 according to the preferred embodiment of FIG. 1. To allow conventional rolled-type dispensing of child's wiping system 100, a series of wiping sheath(s) 104 joined side-edge to side-edge preferably form a continuous band 150 of wiping sheath(s) 104, as shown. Preferably, band 150 is sectioned into individual wiping sheath(s) 104 by a series of perforation lines 152 that extend transversely across band 150, defining a tear line for each wiping sheath 104 (herein embodying at least one perforation structured and arranged to assist the user in detaching at least one hand-wearable cover from such at least one band). Perforation lines 152 preferably consist of alternating bands and perforations that, as in most conventional rolled products, are of approximately uniform spacing and length. The preferred perforations are typically rectangular slits having a transverse orientation relative to the long dimension of band 150, as shown.

FIG. 7 is a perspective view of child's wiping system 100 in rolled configuration 154 according to the preferred embodiment of FIG. 6. As a consumer product, band 150 may preferably form a continuous roll of several hundred individual wiping sheath(s) 104, as shown (herein embodying wherein a plurality of such hand-wearable covers are detachably joined to form at least one continuous band; and wherein such at least one continuous band is in a rolled configuration). Child's wiping system 100 in rolled configuration 154 is preferably coreless and is typically dispensed by end wiping sheath 156, or by center wiping sheath 158 (pulled from the center of the rolled product through a product dispenser).

FIG. 8 is a perspective view of child's wiping system 100 in folded configuration 160 according to the preferred embodiment of FIG. 1. Under appropriate circumstances, child's wiping system 100 may be dispensed from a stack of individual interfolded wiping sheath(s) 164, as shown (herein embodying wherein a plurality of such at least one hand-wearable covers are interfolded to form a dispensable stack).

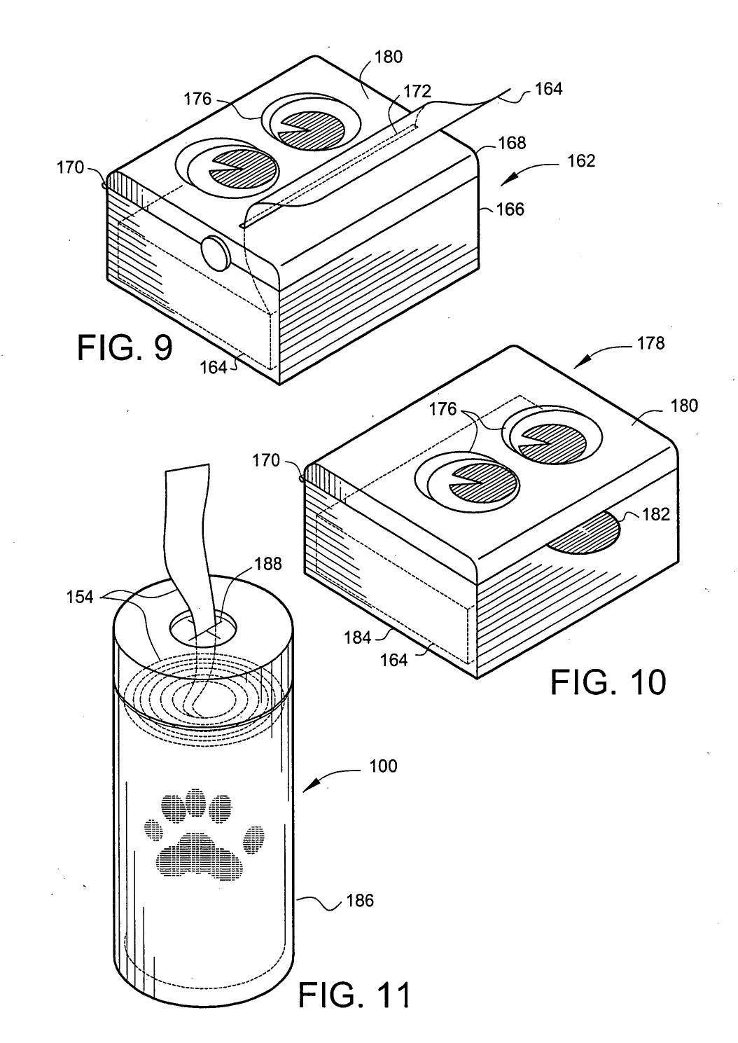

FIG. 9 is a perspective view of folded product dispenser 162 for child's wiping system 100 according to a preferred embodiment of the present invention. Folded product dispenser 162 dispenses individual interfolded wiping sheath(s) 164 from a stack preferably located within the interior of folded product dispenser 162, as shown. Preferably, dispensing an interfolded wiping sheath 164 involves pulling one edge of wiping sheath 164 through slot 172 and away from folded product dispenser 162. The interleaved portion of the interfolded wiping sheath 164 serves to draw up the next sheath, due to the interfacial interaction of the two interfolded wiping sheath(s) 164 at the area of interleave. After the first interfolded wiping sheath 164 has been completely pulled through slot 172, the two interfolded wiping sheath(s) 164 begin to separate. When the two interfolded wiping sheath(s) 164 are completely separated, there should be a sufficient amount of the second interfolded wiping sheath 164 extending outside of slot 172 to allow easy subsequent access, as shown.

Preferably, folded product dispenser 162 has a one piece housing 166 having a lid 168 through which interfolded wiping sheath(s) 164 are dispensed, as shown. Lid 168 is preferably joined to housing 166 by means of an integrally formed hinge 170, as shown. Preferably, slot 172 is sized to allow easy passage of interfolded wiping sheath(s) 164 while limiting air passage in and out of folded product dispenser 162 (to limit evaporative loss pre-moisturizing fluids). Catch 174 is preferably provided to prevent inadvertent opening of lid 168 during operation.

Folded product dispenser 162 is preferably constructed from a lightweight and water-impermeable material, preferably molded plastic. Preferably, folded product dispenser 162 includes playful indicia 110, shown in the embodiment of FIG. 9 as a pair of eyes 176 located on lid 168 above slot 172, as shown. Under appropriate circumstances, other playful and useful design features may be used to allow the packaging of folded product dispenser 162 to be attractive to a small child. Lid 168 is preferably joined to housing 166 by means of an integrally formed hinge 170, as shown. Under appropriate circumstances, other methods of hinging, including applied hinge assemblies, may suffice. Folded product dispenser 162 is preferably refillable, however, under appropriate circumstances, dispenser 162 may be a one-use package.

FIG. 10 is a perspective view of an alternate folded product dispenser 178 for interfolded wiping sheath(s)164 according to the embodiment of FIG. 9. To prevent loss of pre-moisturizing fluid, slot 172 is omitted, allowing interfolded wiping sheath(s) 164 to be fully contained within housing 184 of folded product dispenser 178 until needed. Opening lid 180 permits access to the interior of housing 184 and the interfolded wiping sheath((s), 164 contained within. Tab 182 is preferably added to lid 180, as shown, allowing a small child to open lid 180 with limited effort. Preferably, tab 182 is playfully arranged to appear as a tongue when applied in conjunction with eyes 716, as shown. Under appropriate circumstances, other combinations of playful and useful design features may be used. Lid 180 is preferably joined to housing 184 by means of an integrally formed hinge 170, as shown. Folded product dispenser 178 is preferably constructed from a lightweight molded plastic, such as commercial grade polystyrene. Folded product dispenser 178 is preferably refillable, however, under appropriate circumstances, dispenser 178, may be a one-use package such a disposable travel package.

FIG. 11 is a perspective view of center-pull dispenser 186 for child's wiping system 100 according to another preferred embodiment of the present invention. Also known as center-flow-type dispensers, center-pull dispenser 186 is, under appropriate circumstances, a preferred dispenser for the rolled configuration 154 of child's wiping system 100, as shown. Conventional center-pull products are preferably dispensed through a narrow opening 188 or passage through which the center-pull product is pulled, as shown.

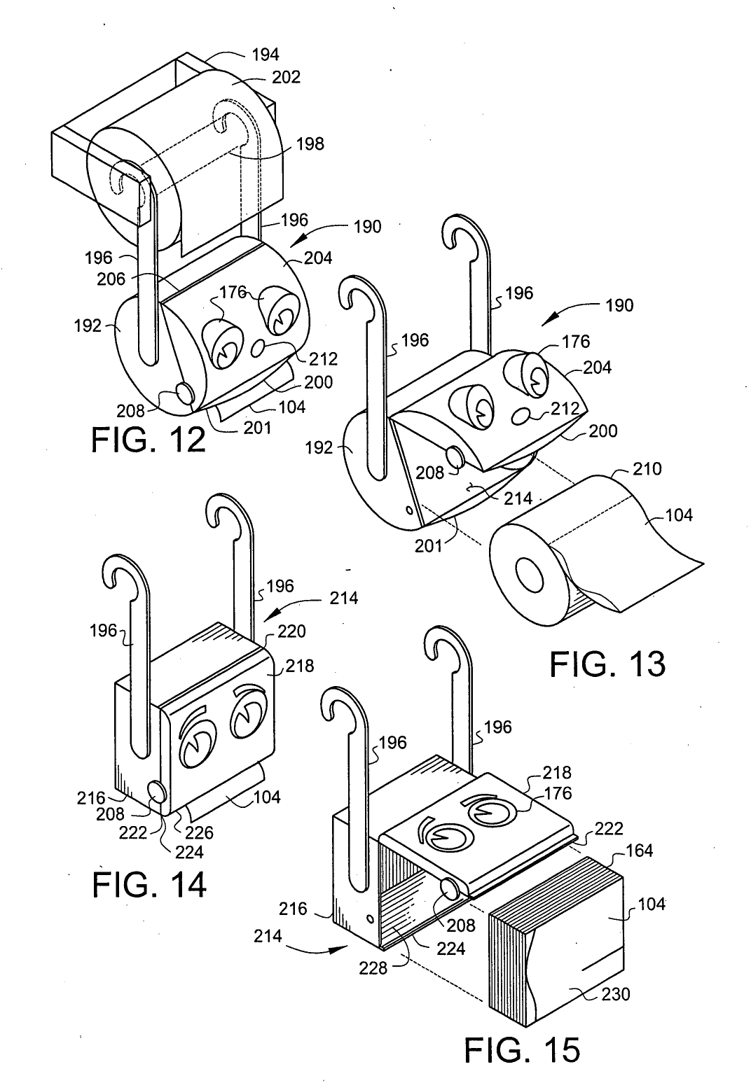

FIG. 12 is a perspective view of hanging dispenser 190 for dispensing child's wiping system 100, adapted to hang from a standard dry tissue dispenser 194 according to a preferred embodiment of the present invention. Hanging dispenser 190 preferably consists of housing 192 having hooked supports 196 firmly mounted to each end, allowing dispenser 190 to be suspended from the roll support shaft 198 of dry tissue dispenser 194, as shown. Preferably, hooked supports 196 are of a sufficient length to position hanging dispenser 190 below dry tissue dispenser 194, allowing dry tissue 202 to be dispensed in a normal fashion, as shown. Preferably, lid 204 is joined to housing 192 with integral hinge 206, as shown. A single catch 208 is preferably mounted to each end of housing 192 to retain lid 204 in a closed position, as shown. A first wiping sheath guide 200 is preferably located longitudinally along the leading edge of lid 204, and a complementary second wiping sheath guide 201 is located along the leading edge of housing 192 opposite first wiping sheath guide 200, as shown. Closing lid 204 brings first wiping sheath guide 200 closely adjacent to second wiping sheath guide 201, creating a frictional path for wiping sheath 104, as shown. Preferably, the gap formed between first wiping sheath guide 200 and second wiping sheath guide 201 is sized to be approximately equivalent to, or somewhat smaller than, the thickness of wiping sheath 104 (about 0.02 inches). A wiping sheath 104 may be dispensed by grasping and pulling the exposed wiping sheath 104 away from hanging dispenser 190, separating wiping sheath 104 from the remaining sheaths on wiping sheath roll 210 (located within housing 192) by tearing along the perforation lines 152 (not illustrated). The process of dispensing wiping sheath 104 leaves a new exposed wiping sheath engaged in the frictional gap formed between wiping sheath guide 200 and wiping sheath guide 201 (herein embodying at least one dispenser structured and arranged to dispense the at least one child's hand-wearable wipe exterior of such housing). Preferably, the dispensing process may be repeated until the supply of wiping sheath(s) 104 on wiping sheath roll 210 is exhausted.

FIG, 13 is a perspective view of hanging dispenser 190 for child's wiping system 100 in an open position to receive wiping sheath roll 210 according to the preferred embodiment of FIG. 12. Opening hanging dispenser 190 allows a single wiping sheath roll 210 to be inserted into interior portion 214 of housing 192, as shown (herein embodying at least one housing having at least one interior portion structured and arranged to house the at least one child's hand-wearable wipe and further embodying at least one portal structured and arranged to allow access to such at least one interior portion). After wiping sheath roll 210 is inserted into hanging dispenser 190, a first wiping sheath 104, located on the outer circumference of wiping sheath roll 210, is placed over second wiping sheath guide 201. Lid 204 is closed, bringing first wiping sheath guide 200 down to contact the first wiping sheath 104. Preferably, catch 208 is used to engage lid 204 to housing 192, firmly securing lid 204 in a closed position. Hanging dispenser 190 is preferably constructed from a lightweight and water-impermeable material (herein embodying at least one moisture retainer structured and arranged to retain moisture within such at least one interior portion), preferably molded plastic, such as polystyrene. Preferably, hanging dispenser 190 includes playful indicia, illustrated in the embodiment of FIG. 12 and FIG. 13 as a pair of eyes 176 and nose 212 located on lid 204 above sheath guide 200, as shown. Under appropriate circumstances, other combinations of playful and useful design features, such as child customizable exterior elements, may be used.

FIG. 14 is a perspective view of hanging dispenser 214 containing a stack of interfolded wiping sheath(s) 164 according to a preferred embodiment of the present invention. Hanging dispenser 214 preferably consists of housing 216 having hooked supports 196 firmly mounted to each end, allowing dispenser 214 to be suspended from the dry tissue dispenser 194 (of FIG. 12). Preferably, lid 218 is joined to housing 216 with integral hinge 220, as shown. A single catch 208 is preferably mounted to each end of housing 216 to retain lid 218 in a closed position, as shown. Preferably, wiping sheath 104 is dispensed through slot aperture 226i formed along edge 222 of lid 218 and edge 224 of housing 216, as shown.

FIG. 15 is a perspective view of hanging dispenser 214 in an open position according to the preferred embodiment of FIG. 14. Interior portion 228 of hanging dispenser 214 is preferably adapted to receive a stack of interfolded wiping sheath(s) 164, as shown. After the stack of interfolded wiping sheath(s) 164 is inserted into hanging dispenser 214, an end portion 230 of first wiping sheath 104 is positioned over edge 224 to prepare hanging dispenser 214 for use. Lid 218 is closed with catch 208, preferably, used to firmly secure lid 218 to housing 216. Hanging dispenser 214 is preferably constructed from a lightweight and water-impermeable material, preferably molded plastic, such as polystyrene (herein embodying at least one moisture retainer structured and arranged to retain moisture within such at least one interior portion). Preferably, hanging dispenser 214 includes playful indicia, illustrated in the embodiment of FIG. 14 and FIG. 15 as a pair of eyes 176 located on lid 218, as shown. Under appropriate circumstances, other combinations of playful and useful design features may be used.

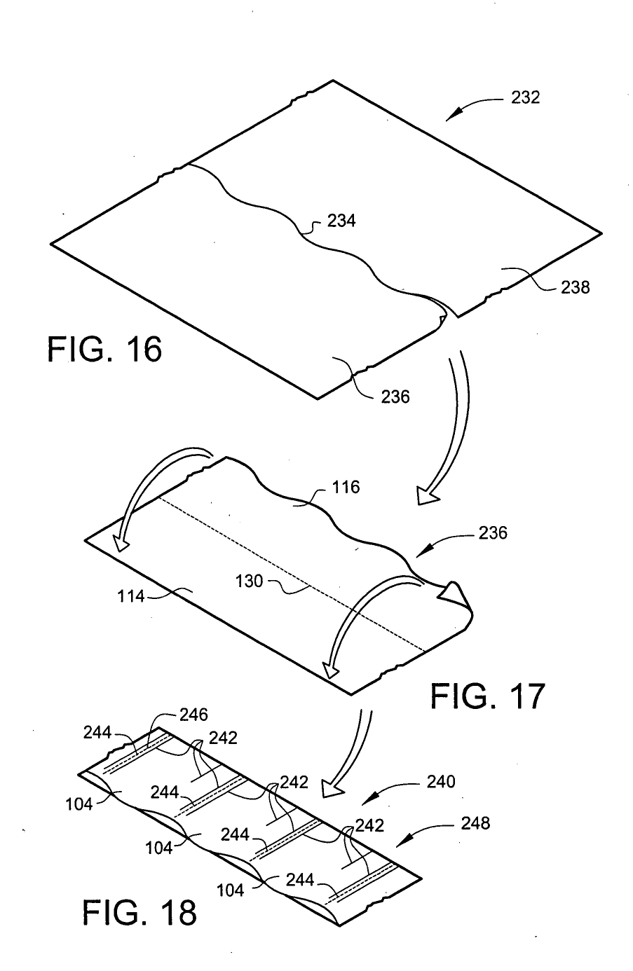

FIG. 16 is a perspective view of a preferred step in the manufacture of child's wiping system 100 according to the embodiment of FIG. 1. It should be noted that the following steps describe, diagrammatically, a preferred method of producing child's wiping system 100. Under appropriate circumstances, a variety of well-known automated methods and mechanisms may be used to accomplish the following steps. Preferably, a continuous serrated cut 234 is made in non-woven sheet 232 that has been pre-cut to allow two identically sized sheets, sheet 236 and sheet 238 to be produced, as shown. Several preferred methods for cutting non-woven fabrics are available. For example, non-woven sheet 232 may be pulled over a rotating drum having a rotating blade. The blade can be stationary, which creates a band with straight edges, or the blade can oscillate to create edges that are non-parallel, as in serrated cut 234. Other preferred methods of cutting non-woven sheet 232 include the use of water jets, lasers and/or ultrasonic cutters. The ultrasonic process is especially versatile allowing cutting, slitting, trimming, tacking, embossing and cutting.

FIG. 17 is a perspective view of another preferred step in the manufacture of child's wiping system 100 according to the embodiment of FIG. 16. Preferably, sheet 236 is folded along top edge 130 to bring front panel 114 in contact with back panel 116, as shown.

FIG. 18 is a perspective view of another preferred step in the manufacture of a child's wipe according to the embodiment of FIG. 17. As shown, seams 242 and perforations 244 are applied to folded sheet 240, prior to application of printing, as shown. If individually folded wiping sheath(s) 104 are to be produced, cuts 246 rather than perforations 244, are made to separate individual wiping sheath(s) 104 from band 248. Prior to, or subsequent to packaging, a moisturizing fluid is preferably used to impregnate the fabric of child's wiping system 100.

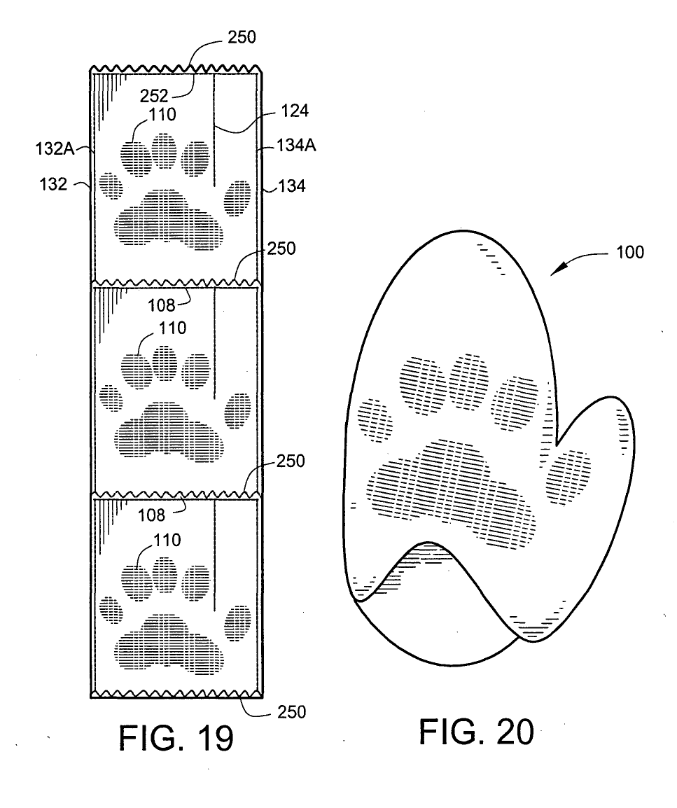

FIG. 19 is a front view of an alternate arrangement of multiple wiping sheath(s) 104 according to the embodiment of FIG. 1. Under appropriate circumstances, it may be preferred to orient wiping sheath(s) 104 within band 150 such that opening 108 is located along perforation line 250, as shown. An additional top seam 252 is preferably applied to wiping sheath 104, as shown.

FIG. 20 is a front view of an alternate preferred embodiment of child's wiping system 100. Under appropriate circumstances, child's wiping system 100 may be configured in a diverse range of shapes, such as the “mitten” shape of FIG. 20.

Although applicant has described applicant's preferred embodiments of this invention, it will be understood that the broadest scope of this invention includes such modifications as diverse shapes and sizes and materials. Such scope is limited only by the below claims as read in connection with the above specification. Further, many other advantages of applicant's invention will be apparent to those skilled in the art from the above descriptions and the below claims.

What is claimed is:

1) A child's disposable wiping system to remove urine and feces after the child's elimination of bodily waste, hand-wearable by the child, comprising in combination:
a) at least one hand-wearable cover structured and arranged to substantially cover a child's hand; and
b) at least one pre-moisturizer structured and arranged to pre-moisturize said at least one hand-wearable cover;
c) wherein said at least one hand-wearable cover is fluid-permeable; and
d) wherein said at least one pre-moisturizer substantially impregnates said at least one hand-wearable cover.

2) The system according to claim 1 wherein said at least one hand-wearable cover comprises at least one surface structured and arranged to retain the bodily waste during wiping.

3) The system according to claim 1 wherein said hand-wearable cover comprises a single layer fabric.

4) The system according to claim 3 wherein said single layer fabric comprises at least one non-woven material.

5) The system according to claim 4 wherein said single layer fabric comprises at least one water dispersible binder.

6) The system according to claim 1 wherein said at least one hand-wearable cover comprises:
a) a first side portion;
b) a second side portion; and
c) at least one opening;
d) wherein said first side portion and said second side portion are joined to define at least one interior portion; and
e) wherein said at least one opening is structured and arranged to permit the child's hand to be removably inserted into said at least one interior portion.

7) The system according to claim 6 wherein said at least one hand-wearable cover further comprises:
a) at least one grip assister structured and arranged to assist in gripping at least one peripheral portion of said at least one opening;
b) whereby said grip assister facilitates separation of said first side portion from said second side portion to assist insertion of the child's hand through said opening of said hand-wearable cover.

8) The system according to claim 7 wherein said at least one grip assister comprises at least one scalloped-shaped edge along said at least one peripheral portion of said at least one opening.

9) The system according to claim 1 wherein said at least one hand-wearable cover comprises at least one finger receiving portion structured and arranged to receive at least one finger of the child's hand.

10) The system according to claim 5 wherein said at least one hand cover comprises a thumb receiving portion structured and arranged to receive a thumb of the child's hand.

11) The system according to claim 1 wherein:
a) a plurality of said hand-wearable covers are detachably joined to form at least one continuous band; and
b) said at least one continuous band is in a rolled configuration.

12) The system according to claim 11 wherein said at least one continuous band comprises at least one perforation structured and arranged to assist the user in detaching at least one hand-wearable cover from said at least one band.

13) The system according to claim 1 wherein a plurality of said at least one hand-wearable covers are interfolded to form a dispensable stack.

14) A method of making at least one child's hand-wearable wipe comprising the steps of:
a) receiving a quantity of desired fabric material;
b) reducing such fabric material to a desired working width;
c) cutting such fabric to produce at least one continuous sheet of a desired width;
d) folding such at least one continuous sheet to form at least one continuous, essentially “U”-shaped band; and
e) seaming such at least one continuous, essentially “U”-shaped band to form at least one child's hand-wearable wipe.

15) The method of making at least one child's hand-wearable wipe according to claim 14 further comprising the step of applying to such at least one continuous, essentially “U”-shaped band, at least one line of perforations located essentially perpendicular to the longitudinal axis of such at least one continuous, essentially “U”-shaped band.

16) The method of making at least one child's hand-wearable wipe according to claim 14 further comprising the step of cutting such at least one continuous, essentially “U”-shaped band to produce at least one child's hand-wearable wipe.

17) The method of making at least one child's hand-wearable wipe according to claim 16 further comprising the step of folding such at least one child's hand-wearable wipe to form a dispensable stack.

18) The method of making at least one child's hand-wearable wipe according to claims 14, 15 or 17 further comprising the step of impregnating such at least one child's hand-wearable wipe with at least one moisturizing fluid.

19) A child's hand-wearable wipe dispenser system adapted to dispense at least one child's hand-wearable wipe comprising:
a) at least one holder structured and arranged to hold at least one child's hand-wearable wipe;
b) wherein said at least one holder comprises:
i) at least one housing having at least one interior portion structured and arranged to house the at least one child's hand-wearable wipe;
ii) at least one dispenser structured and arranged to dispense the at least one child's hand-wearable wipe exterior of said housing;
iii) at least one portal structured and arranged to allow access to said at least one interior portion; and
iv) at least one moisture retainer structured and arranged to retain moisture within said at least one interior portion;
c) wherein the size and configuration of said at least one holder closely conforms to the dispensable format of the at least one child's hand-wearable wipe; and
d) wherein said at least one holder comprises indicia.

20) The system according to claim 19 wherein said at least one holder is supported by a dry rolled tissue dispenser.

21) Each and every novel feature, element, combination, step and/or method disclosed or suggested by this provisional patent application.
